(19) United States
(12) Patent Application Publication
(10) Pub. No.: US 2024/0336635 A1
(43) Pub. Date: Oct. 10, 2024

(54) PROCESS FOR THE PRODUCTION OF (1R,2S,5R)-1-AMINO-5-[2-(DIHYDROXYBORANYL)ETHYL]-2-[(DIMETHYLAMINO)METHYL]-CYCLOHEXANE-1-CARBOXYLIC ACID

(71) Applicant: Molecure S.A., Warsaw (PL)

(72) Inventors: Marta Magdycz, Dabrowa Chotomowska (PL); Lukasz Mucha, Warszawa (PL); Damian Kusmirek, Warszawa (PL); Kamil Lisiecki, Warszawa (PL); Marek Masnyk, Warszawa (PL); Piotr Pomaranski, Brochów (PL); Magdalena Tyszkiewicz, Warszawa (PL); Stanislaw Pikul, Ceradz Dolny (PL); Elzbieta Sobolewska, Ustanów (PL)

(21) Appl. No.: 18/626,052

(22) Filed: Apr. 3, 2024

Related U.S. Application Data
(60) Provisional application No. 63/494,557, filed on Apr. 6, 2023.

(30) Foreign Application Priority Data
Apr. 6, 2023 (PL) P.444342

(51) Int. Cl. C07F 5/02; C07C 45/59

(52) U.S. Cl. CPC C07F 5/025; C07C 45/59

(57) ABSTRACT

The present invention relates to a process for the preparation of (1R,2S,5R)-1-amino-5-[2-(dihydroxyboranyl)ethyl]-2-[(dimethylamino)methyl]cyclohexane-1-carboxylic acid in anhydrous crystalline form A. The present invention further relates to methyl (1R,2R,4R)-2-acetamido-2-(tert-butylcarbamoyl)-4-(2-(4,4,5,5-tetramethyl-1,3,2-dioxaborolan-2-yl)ethyl)-cyclohexane-1-carboxylate which is the intermediate in this process.

### CROSS REFERENCE TO RELATED APPLICATIONS

This application claims the benefit of priority to the Polish Patent Application No. P.444342, filed Apr. 6, 2023, and to the U.S. Provisional Patent Application No. 63/494,557, filed Apr. 6, 2023.

### FIELD OF THE INVENTION

The present invention relates to a process for the preparation of (1R,2S,5R)-1-amino-5-[2-(dihydroxyboranyl)ethyl]-2-[(dimethylamino)methyl]cyclohexane-1-carboxylic acid in anhydrous crystalline form A. The present invention further relates to methyl (1R,2R,4R)-2-acetamido-2-(tert-butylcarbamoyl)-4-(2-(4,4,5,5-tetramethyl-1,3,2-dioxaborolan-2-yl)ethyl)-cyclohexane-1-carboxylate which is the intermediate in this process.

### BACKGROUND OF THE INVENTION

(1R,2S,5R)-1-Amino-5-[2-(dihydroxyboranyl)ethyl]-2-[(dimethylamino)methyl]-cyclohexane-1-carboxylic acid of structural formula 1 is described as Example 38 in international patent publication No. WO2017/191130 A2, US patent publication No. 10,391,077 B2, and US patent publication No. 10,912,755 B2, all claiming priority to Polish patent application No. P-417066, filed May 4, 2016, U.S. provisional application No. 62/331,550, filed May 4, 2016, and U.S. provisional application No. 62/444,669, filed Jan. 10, 2017, the disclosures of which are hereby incorporated by reference in their entirety.

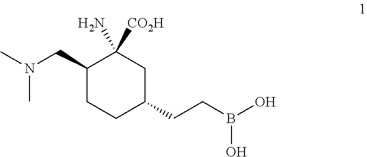 [SMILES: CN(C)C[C@@H]1CC[C@@H](CCB(O)O)C[C@]1(N)C(=O)O]

The compound of formula 1 is a dual inhibitor of both ARG1 and ARG2 enzymes. Such compounds might be used in a treatment of disorders associated with overexpression of those enzymes. The relevant disorders are, e.g., colorectal cancer, ovarian cancer, renal cell carcinoma, pancreatic ductal adenocarcinoma, and acute myeloid leukemia.

Further treatable disorders, diseases, or conditions comprise cardiovascular disorders, sexual disorders, wound healing disorders, gastrointestinal disorders, autoimmune disorders, immune disorders, human immunodeficiency virus (HIV) infection, hepatitis B virus (HBV) infection, hepatitis C virus (HCV) infection, Helicobacter pylori infections, pulmonary disorders, hemolytic disorders, and neoplastic diseases.

The neoplastic disease is a cancer selected from the group consisting of gastric cancer, colorectal cancer, pancreatic cancer, liver cancer, breast cancer, lung cancers, renal cell carcinoma, prostate carcinoma, multiple myeloma, acute and chronic leukemias, T cell, B cell and NK cell lymphomas, brain tumors, squamous-cell carcinomas of the head and neck, and melanoma.

The above-mentioned WO and US patent documents describe the synthesis of the compound of formula 1 from ethyl (4R)-2-hydroxy-4-vinylcyclohex-1-ene-1-carboxylate of formula 2. This synthesis is outlined in Scheme 1 below.

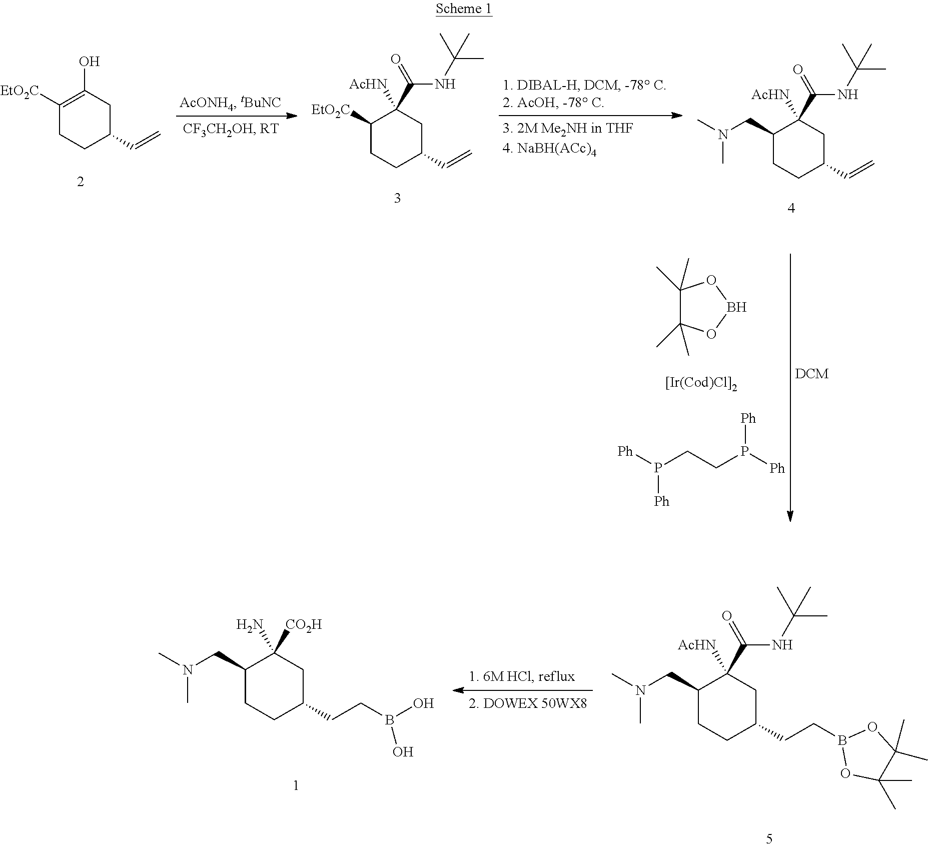

The compound of formula 2 is reacted with ammonium acetate and tert-butyl isocyanide in 2,2,2-trifluoroethanol at ambient temperature. After stirring overnight, the crude product 3 and its diastereoisomer are isolated by extraction and the major diastereoisomer 3 is purified by column chromatography. Pure ethyl (1R,2R,4R)-2-acetamido-2-(tert-butylcarbamoyl)-4-vinylcyclohexane-1-carboxylate 3 is then reacted with 1 M DIBAL-H in DCM, glacial acetic acid, 2 M solution of dimethylamine in THF and sodium triacetoxyborohydride to give the crude (1R,2S,5R)-1-acetamido-N-(tert-butyl)-2-[(dimethylamino)methyl]-5-vinylcyclohexanecarboxamide of formula 4. The crude product is purified via column chromatography to give the corresponding product as a single diasteroisomer. In the next synthetic step, pure 4 is transformed into (1R,2S,5R)-1-acetamido-N-(tert-butyl)-2-[(dimethylamino)methyl]-5-(2-(4,4,5,5-tetramethyl-1,3,2-dioxaborolan-2-yl)ethyl)cyclohexane-1-carboxamide of formula 5 by using 1,2-bis(diphenylphosphino)ethane, bis(1,5-cyclooctadiene)diiridium(I) dichloride and 4,4,5,5-tetramethyl-1,3,2-dioxaborolane in DCM. The pure product 5 is obtained by purification via column chromatography. Finally, the compound of formula 5 is subjected to a reaction of deprotection of amine, carboxylic and boronic acid moieties. In this step, 6 M HCl and reflux are applied, the crude product obtained as a hydrochloride salt is then desalted with DOWEX® 50WX8 using 0.1 M ammonia in water as an eluent. The desired product 1 is obtained as a white solid.

The above-presented synthesis of the compound of formula 1 requires at least three chromatographic purifications. This method of isolation requires large amounts of solvents, it is time consuming and cannot be considered suitable for scaling up. Moreover, in the penultimate step, a heavy-metal based catalyst bis(1,5-cyclooctadiene)diiridium(I) dichloride is used, and this may cause problem with residual iridium in the final material. There is no information if the product 1 is obtained in crystalline form. The above features suggest that the presented process cannot be considered efficient and scalable.

### SUMMARY OF THE INVENTION

The present invention provides a process for the preparation of methyl (1R,2R,4R)-2-acetamido-2-(tert-butylcarbamoyl)-4-(2-(4,4,5,5-tetramethyl-1,3,2-dioxaborolan-2-yl)ethyl)-cyclohexane-1-carboxylate of formula 11 in a solid crystalline state.

The present invention provides an industrially scalable process for the preparation of (1R,2S,5R)-1-amino-5-[2-(dihydroxyboranyl)ethyl]-2-[(dimethylamino)methyl]cyclohexane-1-carboxylic acid of formula 1. The process avoids using heavy metal based catalysts and includes only one filtration through the silica gel column chromatography. The final product is obtained in a repeatable manner in terms of the polymorphic form, purity and chiral purity, and meets high standards for drug substance.

The present invention provides a process for the preparation of the solid crystalline compound of formula 1 in anhydrous form A.

The present invention relates to solid crystalline (1R,2S,5R)-1-amino-5-[2-(dihydroxyboranyl)ethyl]-2-[(dimethylamino)methyl]cyclohexane-1-carboxylic acid of formula 1 in anhydrous form A, that is characterized by at least one of the following:
(i) XRPD peaks at 7.39, 8.55, 9.49, 12.83, 14.71, 16.86, 17.37, 17.88, 19.11, 19.88, 20.55, 21.19, 22.18, 22.87, 23.66, 24.52, 25.73, 26.09, 26.94, 28.19, 28.61, 28.88, 29.69, 30.28, 31.45, 32.02, 33.06, 33.36, 34.05, 34.52, 35.15, 36.20, 37.52, 38.95, 40.54, 41.76;
(ii) IR bands at 3134, 2992, 2918, 2859, 2826, 2789, 2764, 2727, 1584, 1522, 1458, 1404, 1369, 1344, 1310, 1263, 1202, 1159, 1105, 1074, 1028, 993, 887, 845, 762, 731 cm⁻¹;
(iii) DSC trace with two broad endothermic events: onset at 141.9° C. and peak at 173.7° C.; onset at 237.4° C. and peak at 254.1° C.

The aforementioned characteristics are substantially in accordance to FIGS. 1 to 3.

### BRIEF DESCRIPTION OF DRAWINGS

FIG. 1. XRPD diffractogram of (1R,2S,5R)-1-amino-5-[2-(dihydroxyboranyl)ethyl]-2-[(dimethylamino)methyl]cyclohexane-1-carboxylic acid in anhydrous form A.

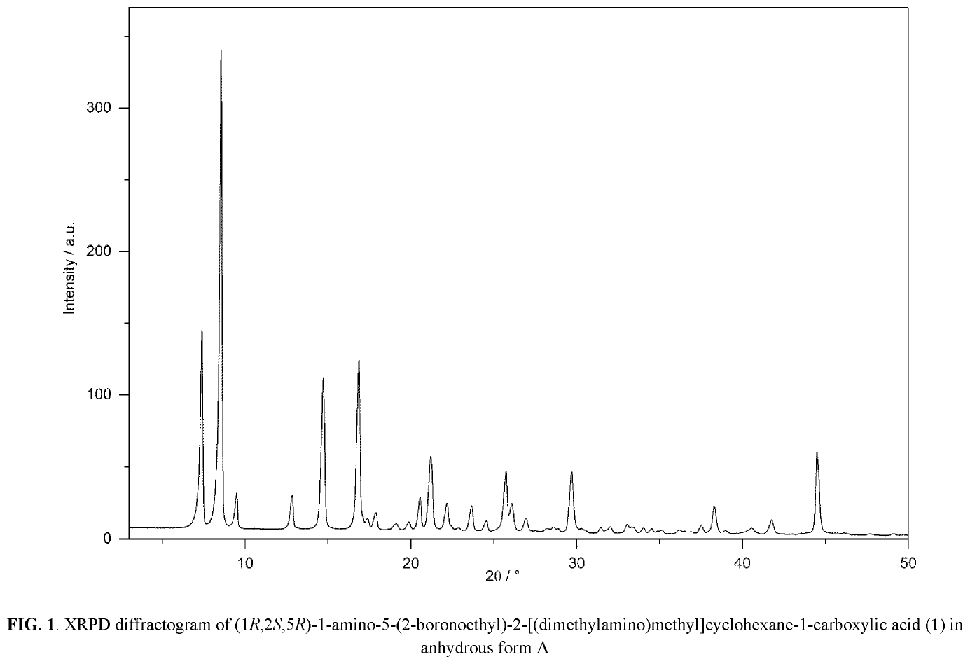

FIG. 2. FT-IR (ATR) spectrum of (1R,2S,5R)-1-amino-5-[2-(dihydroxyboranyl)ethyl]-2-[(dimethylamino)methyl]cyclohexane-1-carboxylic acid in anhydrous form A.

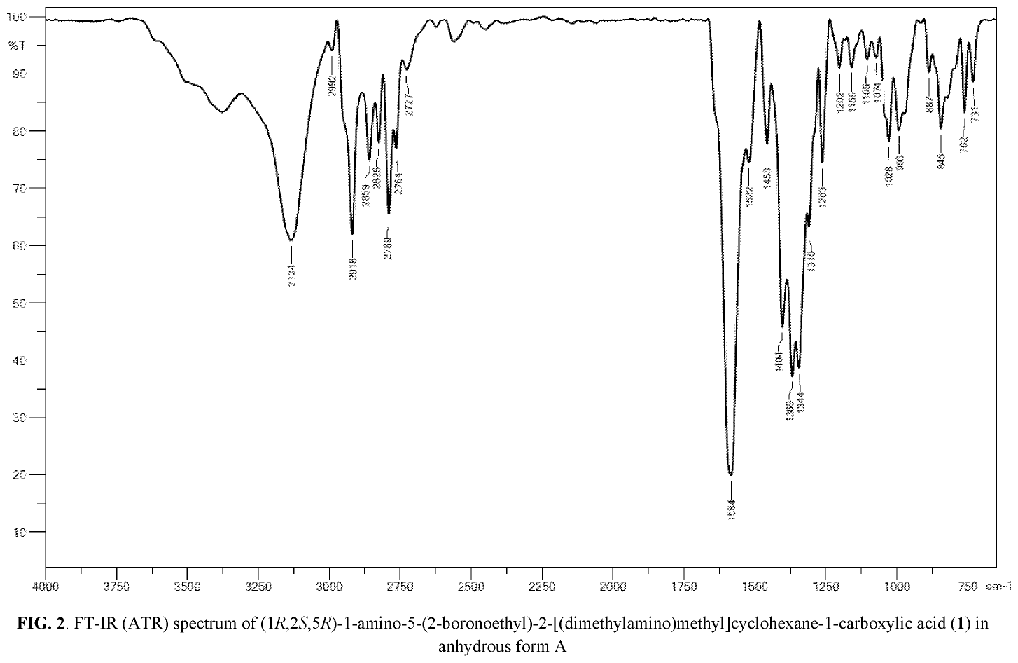

FIG. 3. DSC thermogram of (1R,2S,5R)-1-amino-5-[2-(dihydroxyboranyl)ethyl]-2-[(dimethylamino)methyl]cyclohexane-1-carboxylic acid in anhydrous form A.

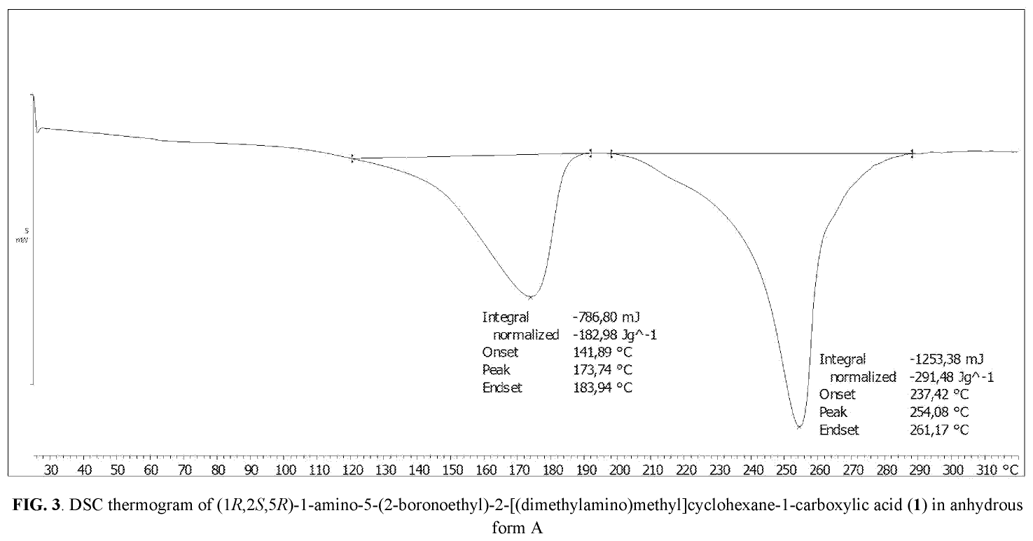

FIG. 4. ¹H NMR (D₂O at 400 MHz) spectrum of (1R,2S,5R)-1-amino-5-[2-(dihydroxyboranyl)ethyl]-2-[(dimethylamino)methyl]cyclohexane-1-carboxylic acid.

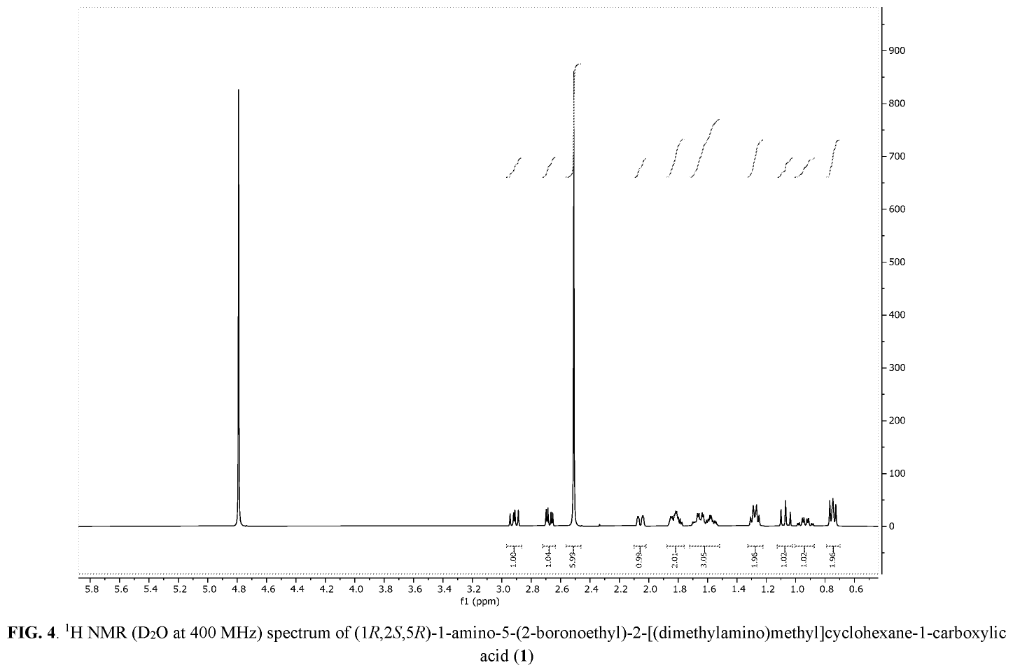

FIG. 5. ¹³C NMR (D₂O at 100 MHz) spectrum of (1R,2S,5R)-1-amino-5-[2-(dihydroxyboranyl)ethyl]-2-[(dimethylamino)methyl]cyclohexane-1-carboxylic acid.

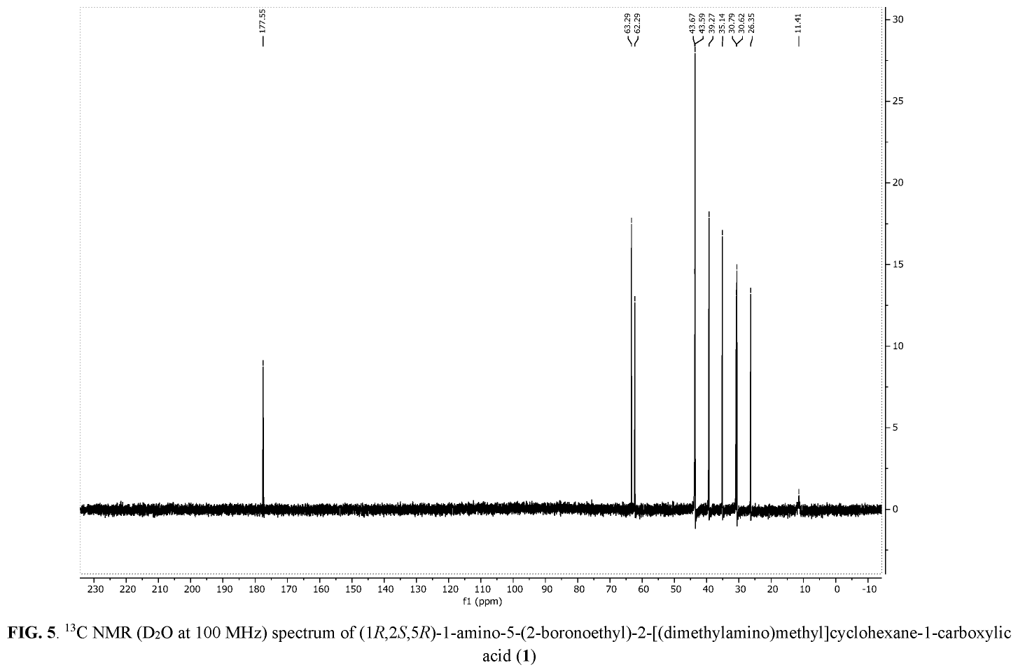

### DETAILED DESCRIPTION OF THE INVENTION

The present description provides efficient and scalable synthesis of methyl (1R,2R,4R)-2-acetamido-2-(tert-butylcarbamoyl)-4-(2-(4,4,5,5-tetramethyl-1,3,2-dioxaborolan-2-yl)ethyl)cyclohexane-1-carboxylate of formula 11. This compound was never isolated nor described yet.

The compound of formula 6 was prepared according to the literature protocol (Jiricek, J.; Blechert, S. Enantioselective Synthesis of (−)—Gilbertine via a Cationic Cascade Cyclization. J. Am. Chem. Soc. 2004, 126, 3534-3538). The compound 6 is reacted with bromine and triphenylphosphine, in the presence of organic base, preferably pyridine, in a solvent, preferably DCM, at room temperature. Imidazole could also be used as the organic base. After obtaining full conversion of substrate, an alcohol, preferably methanol, is added to quench the reaction. The crude compound 7 is isolated by washing with water, followed by a mild base, preferably NaHCO₃ and Na₂S₂O₃, and precipitation of triphenylphosphine with the use of non-polar solvent, preferably an alkane, more preferably n-hexane. After evaporation of solvents, the crude compound 7 is used in the next step without any further purification.

The obtained material of formula 7 is dissolved in a solvent, preferably acetonitrile, and aqueous HCl is added at ambient temperature. After obtaining full conversion of substrate, the solvent is removed in vacuo and aqueous phase is extracted with solvent, preferably ether, more preferably MTBE. Organic phase is washed with a mild base, preferably saturated aqueous NaHCO₃, and brine. After evaporation of solvents, the crude compound 8 is used in the next step without any further purification.

In the next step, crude compound 8 in a solvent, preferably DMF, is added at ambient temperature to the mixture of Ph₃P, B₂(pin)₂, CuI and MeOLi suspended in solvent, preferably DMF. The reaction was carried out at elevated temperature, preferably 40° C. When full conversion of the substrate is obtained, the solution is filtered through the Celite pad and solvent, preferably ether, more preferably MTBE, and 1 M HCl is added. After phase separation, aqueous phase is extracted with a solvent, preferably ether, more preferably MTBE and organic phase is washed with brine. After solvents evaporation, triphenylphosphine is precipitated with the use of non-polar solvent, preferably an alkane, more preferably n-hexane. After filtration and solvent evaporation crude compound 9 is purified by distillation under reduced pressure.

The compound of formula 9 is reacted with dimethyl carbonate, in the presence of a strong base, preferably sodium hydride (free from mineral oil), in a solvent, preferably DMF, at elevated temperature, preferably 75° C. It is crucial to maintain the above-mentioned temperature, because higher temperatures cause a decrease in overall purity of the product. When full conversion of the substrate is obtained, the reaction is quenched with saturated aqueous NH₄Cl at ambient temperature. This step is highly exothermic, so quenching is performed slowly. The crude compound of formula 10 is isolated by extraction with a solvent, preferably MTBE and organic phase is washed with water. After evaporation of solvents, the crude compound 10 is used in the next step without any further purification.

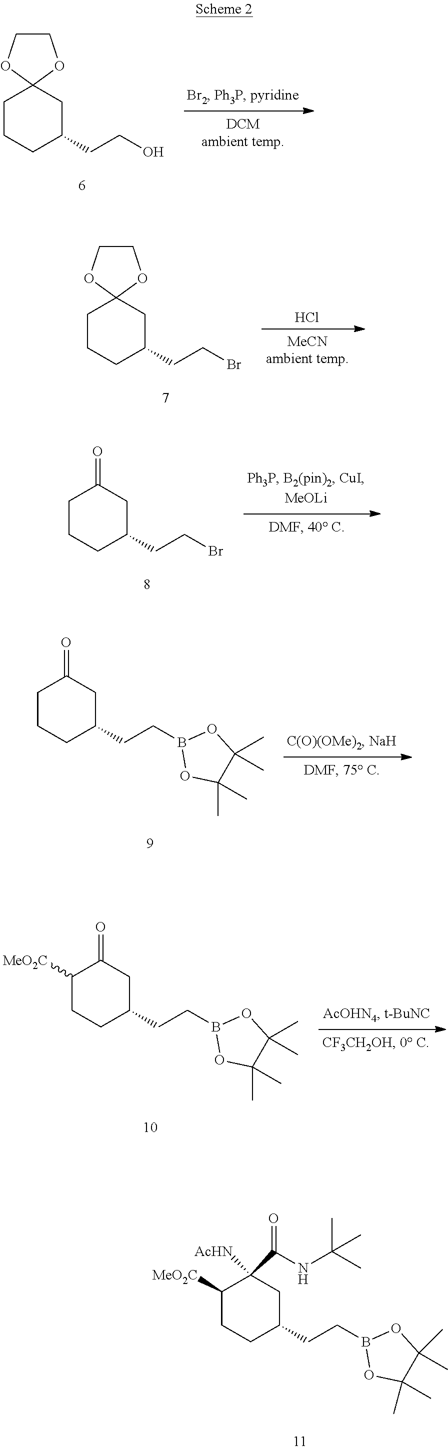

The obtained material of formula 10 is dissolved in a solvent, preferably 2,2,2-trifluoroethanol, and ammonium acetate is added at ambient temperature. After complete dissolution of ammonium acetate, the reaction mixture is cooled to lower temperature, preferably 0° C., and tert-butyl isocyanide is added. After obtaining full conversion, the reaction mixture is concentrated in vacuo and evaporated with a non-polar solvent, preferably an alkane, more preferably n-hexane or n-heptane. To remove excess of ammonium acetate and facilitate crystallization, the obtained suspension is filtered through a short silica gel column. The desired compound is eluted with the solvents, preferably a MTBE/DCM mixture. The fractions which contain the product of formula 11 are evaporated to give the crude compound 11. To remove impurities generated in this step, the crude product is crystalized from a solvent, preferably an ether, more preferably i-Pr₂O. The major impurities are the diastereomers 12, 13, 14, and the diastereomeric products of general formula 15 from the Passerini side reaction. To improve overall yield of this step, mother liquors can be evaporated and purified via column chromatography on silica gel. The obtained material can be further purified via crystallization similar to that described above.

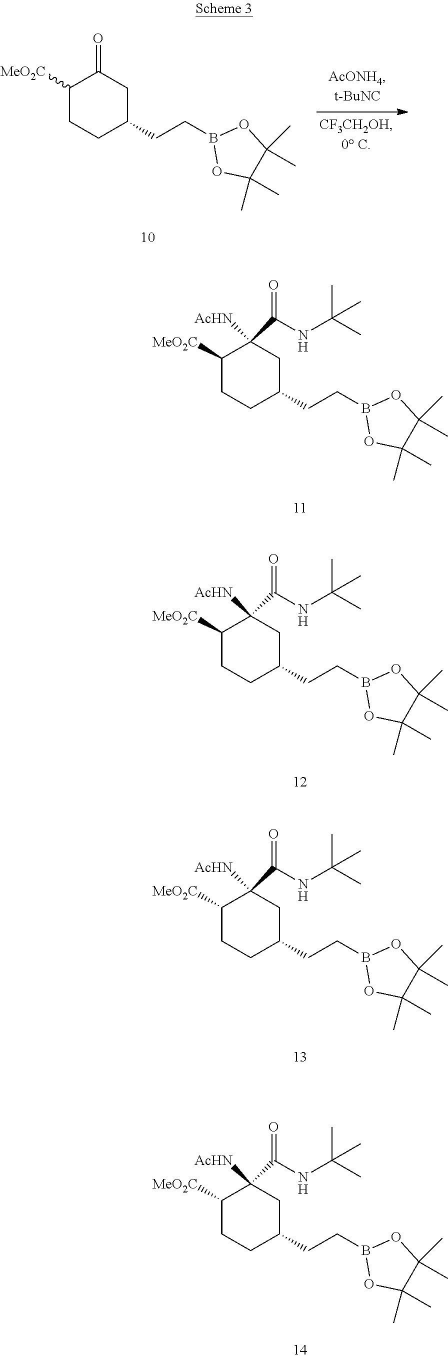

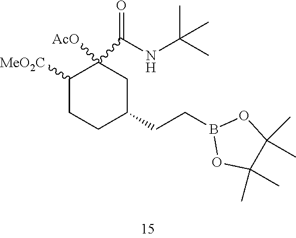

The present invention further provides a method for the synthesis of (1R,2S,5R)-1-amino-5-[2-(dihydroxyboranyl)ethyl]-2-[(dimethylamino)methyl]cyclohexane-1-carboxylic acid of formula 1 in anhydrous form A in a solid crystalline state. Compared to the original approach as outlined in Scheme 1 in the “Background of the invention” section, various changes were introduced into the synthetic procedure in order to improve the yield and purity, making it scalable.

The intermediate compound 5 can be prepared by the reaction of methyl (1R,2R,4R)-2-acetamido-2-(tert-butylcarbamoyl)-4-(2-(4,4,5,5-tetramethyl-1,3,2-dioxaborolan-2-yl)-ethyl)cyclohexane-1-carboxylate of formula 11 with DIBAL-H (in DCM) at a reduced temperature, preferably at −75° C. Low temperature is important because it decreases the amount of over-reduction product of formula 16. During this step reduction of carbonyl moiety in amide group also occurs and impurity of formula 17 is generated. The reaction mixture is quenched with an organic acid, preferably acetic acid (AcOH). During this step large amounts of foam are generated when AcOH is added directly to reaction mixture. When the way of addition is reversed, quenching is much faster, and in consequence less amount of impurity of formula 18 is generated. The crude mixture is further reacted with dimethylamine at a reduced temperature, preferably at −75° C. Then, the reaction mixture is slowly warmed up, preferably to −20° C., and the reducing agent, preferably sodium triacetoxyborohydride, is added. The crude (1R,2S,5R)-1-acetamido-N-(tert-butyl)-2-[(dimethylamino)methyl]-5-(2-(4,4,5,5-tetramethyl-1,3,2-dioxaborolan-2-yl)ethyl)cyclohexane-1-carboxamide of formula 5 is obtained after quenching with saturated aqueous sodium carbonate, and extraction with a solvent, preferably DCM.

The aforementioned transformation can be also performed under flow conditions. In order to do that, the reducing agent, preferably sodium triacetoxyborohydride, is suspended in a solvent, preferably DCM, and cooled to lower temperature, preferably −75° C. To the resulting suspension, a stream of combined reagents—11, dimethylamine (in THF), an organic acid, preferably acetic acid, and DIBAL-H (in DCM), cooled to lower temperature, preferably 0° C., is fed. The reaction mixture is warmed up, preferably to ambient temperature. The crude (1R,2S,5R)-1-acetamido-N-(tert-butyl)-2-[(dimethylamino)methyl]-5-(2-(4,4,5,5-tetramethyl-1,3,2-dioxaborolan-2-yl)ethyl)cyclohexane-1-carboxamide of formula 5 is obtained after quenching with saturated aqueous sodium carbonate, filtration through Celite, and extraction with a solvent, preferably DCM.

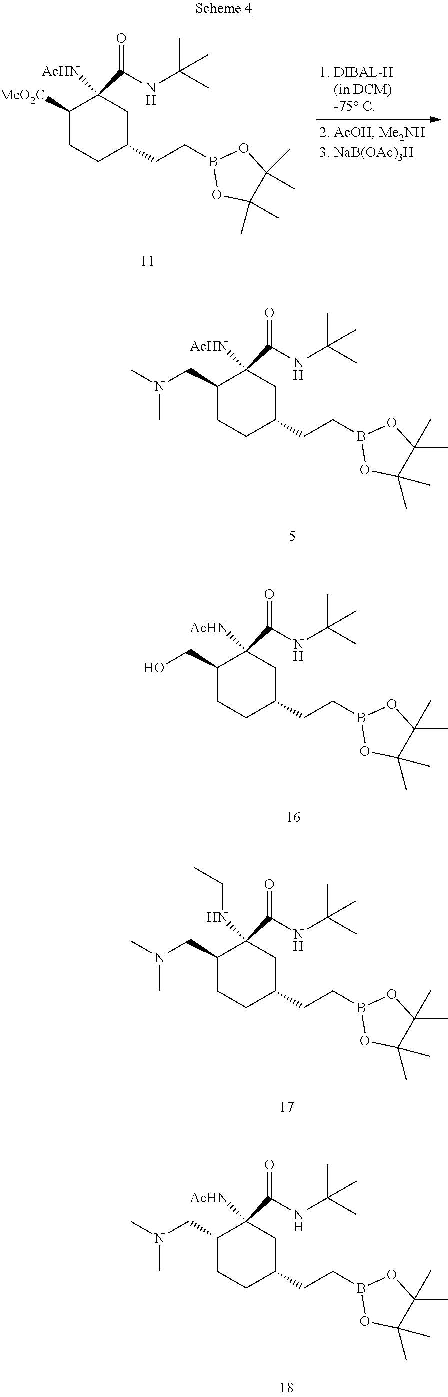

During the reductive amination of the compound of formula 11, various impurities are generated. Before the next step, the crude product mixture containing 5 is dissolved in a solvent, preferably an ether, more preferably MTBE, and the compounds which contain an amino moiety, together with the compound of formula 25, are extracted to an aqueous layer by using 0.5 M HCl. This treatment results in formation of the hydrochloride 19 from the amino compound 5. At the same time, partial deprotection of boronic ester occurs, which leads to generation of deprotected derivatives such as 20, 22, 24, and 25. The crucial impurities, such as the unreacted substrate 11 and the alcohol of formula 16 (formed as a product of over-reduction of carboxylic acid ester moiety), are removed by this extraction.

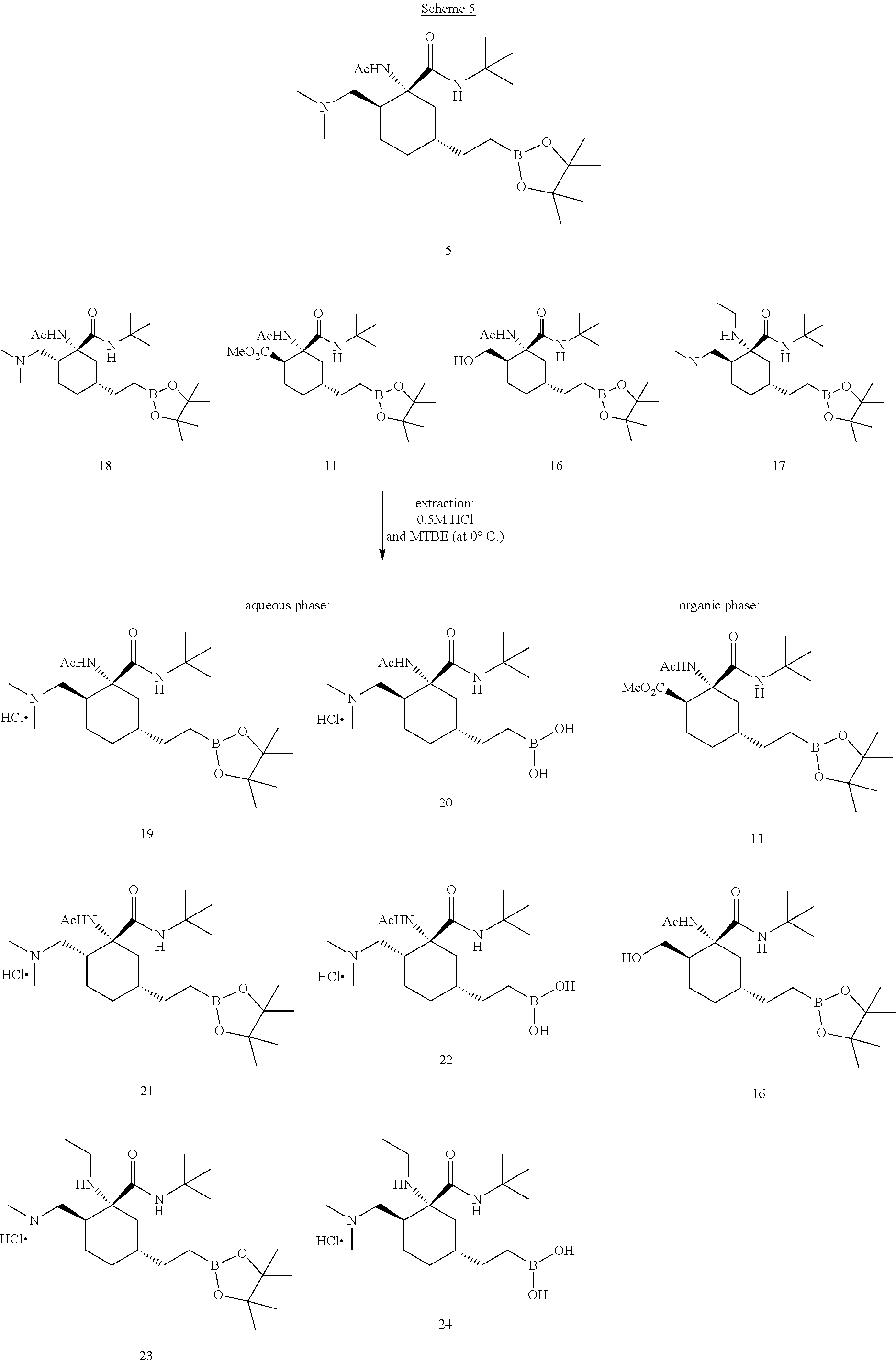

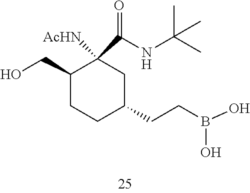 [SMILES: CC(=O)N[C@]1(C(=O)NC(C)(C)C)C[C@H](CCB(O)O)CC[C@H]1CO]

In the next step, 12 M HCl is added to the combined aqueous layers from the aforementioned extraction to obtain final concentration of 6 M HCl. The mixture is then stirred at reflux until full conversion of (1R,2S,5R)-1-acetamido-N-(tert-butyl)-2-[(dimethylamino)-methyl]-5-(2-(4,4,5,5-tetramethyl-1,3,2-dioxaborolan-2-yl)ethyl)cyclohexane-1-carboxamide hydrochloride of formula 19 into the deprotected target compound 1 in the form of its hydrochloride. Under these conditions, the impurities generated in the previous steps are hydrolyzed to give new compounds such as 26, 27 and 28. The deprotected alcohol of formula 25 is converted into the lactone 29. The reaction mixture is cooled down to lower temperature, preferably ambient temperature, and evaporated in vacuo. The obtained crude mixture containing the product 1 in the form of hydrochloride salt is dissolved in H₂O, and loaded onto ion-exchange resin column which is prepared preferably from DOWEX® 50WX8 and regenerated with 1 M HCl. The column is eluted with water until pH=7 is achieved. Then, the product is eluted with 1 M aqueous ammonia. The fractions containing 1 are combined and evaporated to give the crude product.

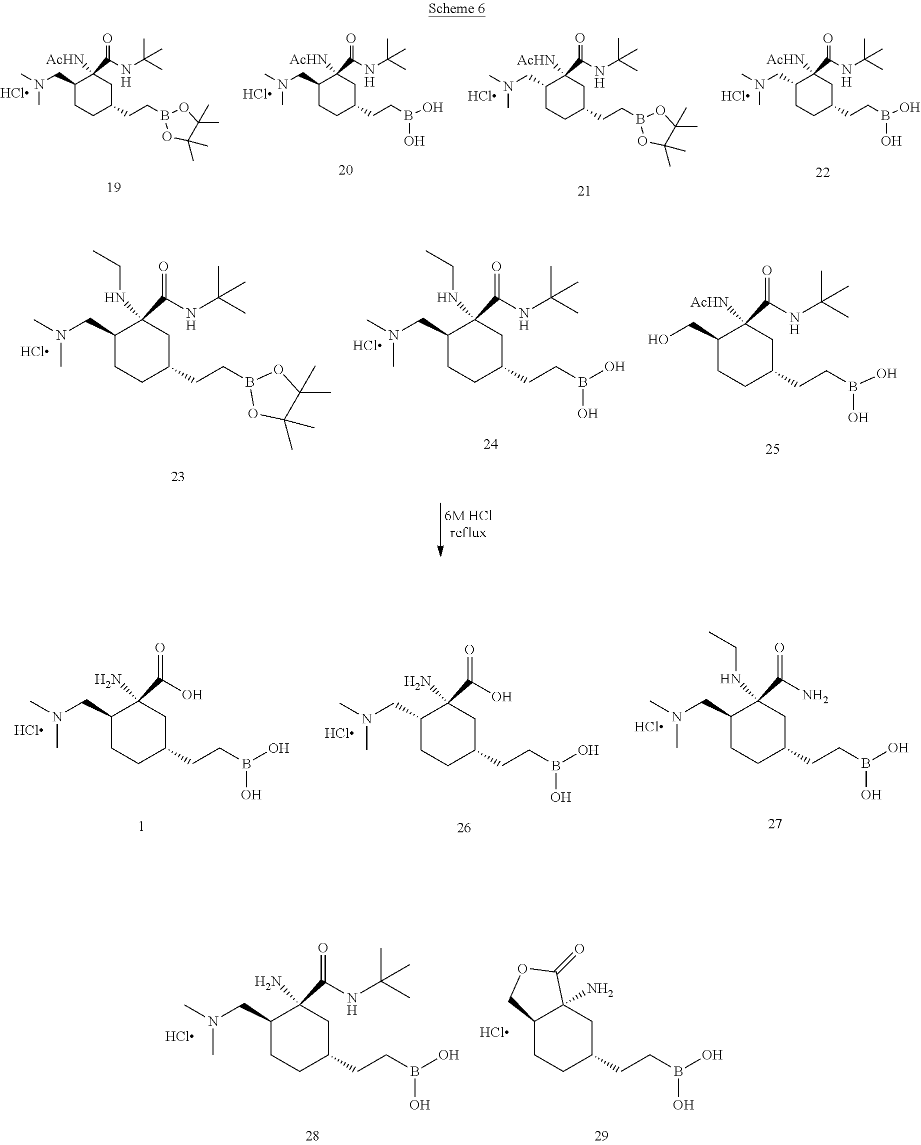

After desalting step, the crude mixture is purified by crystallization. For this purpose the crude mixture is dissolved in a solvent, preferably water, at elevated temperature, preferably at 50° C. Then, an antisolvent, preferably acetone, is added at elevated temperature, preferably at 50° C. The formed suspension is then cooled to lower temperature, preferably 0° C. The suspension is filtered off, washed with a solvent, preferably acetone, and dried to yield a white solid having a purity of >97% by HPLC.

In order to further improve the purity of the material, recrystallization is used. For this purpose the crude product is dissolved in a mixture of solvents, preferably methanol and water, at elevated temperature, preferably at 65° C. The mixture is then concentrated at elevated temperature, preferably at 85° C., to approximately ½ of initial volume, and a portion of solvent, preferably acetone is added. The mixture is concentrated again to ½ of initial volume, a portion of solvent, preferably acetone is added and the whole operation is repeated once more. To the obtained mixture fresh portion of solvent, preferably acetone is added and the obtained suspension is then concentrated to 9/10 of initial volume. Acetone is added and the mixture is allowed to cool down to ambient temperature. The created suspension is then cooled to lower temperature, preferably to 0° C. The crystals are filtered off, and washed with a solvent, preferably acetone. The crystals are collected and dried to give the final product of formula 1 as white crystals with a purity of >99% by HPLC.

During the synthesis of the compound of formula 1, oxidative deboronation occurs, which generates the alcohol compound (1R,2S,5R)-1-amino-2-[(dimethylamino)methyl]-5-(2-hydroxyethyl)cyclohexane-1-carboxylic acid of formula 30, which is mostly removed during the aforementioned crystallizations.

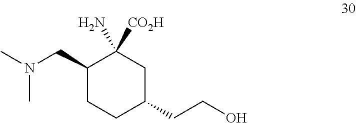 [SMILES: CN(C)C[C@@H]1CC[C@@H](CCO)C[C@]1(N)C(=O)O]

### EXAMPLES

The invention will be illustrated by the following examples that are provided for illustrative purposes only and not intended to limit the scope of the invention in any way.

### Abbreviations

The abbreviations used within this specification have their ordinary meanings.
ATR—Attenuated Total Reflectance
B₂(pin)₂—bis(pinacolato)diboron
DCM—dichloromethane
DIBAL-H—diisobutylaluminum hydride
DMF—dimethylformamide
i-Pr₂O—diisopropyl ether
MeCN—acetonitrile
MTBE—methyl tert-butyl ether
THF—tetrahydrofuran.

### General Procedures

All solvents, substrates and reagents that were commercially available were used without further purification.

NMR spectra were recorded on Agilent Mercury 400 MHz spectrometer.

NMR spectra were recorded in the indicated deuterated solvents that were commercially available.

Resonances are given in parts per million relative to residual solvent (δ 4.79 ppm for D₂O or δ 7.26 ppm for CDCl₃) for ¹H NMR, or to solvent (δ 77.16 ppm for CDCl₃) for ¹³C NMR. Data are reported as follows: chemical shift (6), multiplicity (s=singlet, d=doublet, t=triplet, q=quartet, m=multiplet, br=broad), coupling constants (J in Hz) and integration.

FT-IR spectra were recorded with Shimadzu IRTracer-100 in ATR mode (zinc selenide crystal).

XPRD diffractograms of compound 1 were recorded with Bruker D8 Discover powder X-ray diffractometer equipped with CuKα radiation (1.54 Å) and Vantec detector. The samples were analyzed in a continuous mode with step size of 0.012222760 and step time of 0.9 s over an angular range of 3-50° 2θ.

DSC thermograms were recorded with Mettler Toledo DSC 3 at 5° C./min. gradient.

HPLC/UHPLC purities are expressed by area-under-the-curve %.

Optical rotation was recorded with Perkin Elmer 241 Polarimeter at 20° C. and 589 nm.

The purity of intermediate compound 9 was measured by GC-FID method. The analysis was performed on Phenomenex Zebron ZB-5 ms column (Phenomenex Zebron ZB-5 ms, 0.25 mm×30 m×0.25 μm) and helium was used as a carrier gas with 1 mL/min flow. The following oven program was applied: initial temperature 50° C., ramp 50° C.-310° C. (at 10° C./min), hold at 310° C. for 2 min. Injector temperature was 250° C. and injection volume 1 μL with split ratio 1:50. FID detector temperature was 300° C. and FID air flow 400 mL/min. Run time of the method was 28 min.

The purities of intermediate compounds 11 and 5 were measured by HPLC-UV method. The analysis was performed on Zorbax Extend C18 analytical column (Agilent Zorbax Extend C18, 4.6 mm×150 mm; 3.5 μm) at 30° C., and at mobile phase flow rate of 2 mL/min. The mobile phase is a mixture of solvent A (950 mL water, 50 mL acetonitrile, 840 mg sodium bicarbonate) and solvent B (200 mL water, 800 mL acetonitrile, 840 mg sodium bicarbonate). Elution is carried out under gradient conditions (at 25% of solvent B from 0.0 to 4.0 min, then from 25% to 100% of solvent B from 4.0 to 23.0 min, then at 100% of solvent B from 23.0 to 24.0 min, then from 100% to 25% of solvent B from 24.0 to 25.0 min, then at 25% of solvent B from 25.0 to 33.0 min). Peaks are recorded using UV detection at 210 nm.

The purity of 1 was measured by UHPLC-FLD method using post column derivatization. The analysis was performed on XSelect CSH C18 analytical column (Waters XSelect CSH C18, 3 mm×100 mm; 2.5 μm) at 30° C., and at mobile phase flow rate of 0.5 mL/min. The mobile phase is a mixture of solvent A (1000 mL water, 0.5 g ammonium bicarbonate, adjusted to pH=8.4 with ammonia) and solvent B (200 mL water, 800 mL acetonitrile, 0.5 g ammonium bicarbonate, adjusted to pH=8.4 with ammonia). The derivatization solution is solvent C (1000 mL methanol, 168 mg alizarin) with flow rate of 0.5 mL/min. Elution is carried out under gradient conditions (from 0% to 6% of solvent B from 0.0 to 2.0 min, then at 6% of solvent B from 2.0 to 4.0 min, then from 6% to 10% of solvent B from 4.0 to 5.0 min, then at 10% of solvent B from 5.0 to 9.0 min, then from 10% to 100% of solvent B from 9.0 to 18 min, then from 100% to 0% of solvent B from 18 to 18.10 min, then at 0% of solvent B from 18.10 to 23 min). Peaks are recorded using fluorescence detection at 440 nm for excitation and 580 nm for emission.

The content of impurity of formula 30 was measured by HPLC/UHPLC-UV method. The analysis was performed on Zorbax Extend C18 analytical column (Agilent Zorbax Extend C18, 2.1 mm×100 mm; 3.5 μm) at 30° C., and at mobile phase flow rate of 0.5 mL/min. The mobile phase is a mixture of solvent A (1000 mL water, 840 mg sodium bicarbonate) and solvent B (200 mL water, 800 mL acetonitrile, 840 mg sodium bicarbonate). Elution is carried out under gradient conditions (from 0% to 5% of solvent B from 0.0 to 2.0 min, then at 5% of solvent B from 2.0 to 5.0 min, then from 5% to 7% of solvent B from 5.0 to 8.0 min, then from 7% to 100% of solvent B from 8.0 to 20 min, then from 100% to 0% of solvent B from 20 to 20.10 min, then at 0% of solvent B from 20.10 to 30 min). Peaks are recorded using UV detection at 210 nm.

Reaction yields are expressed by mole %.

### Example 1

### Preparation of (R)-7-(2-bromoethyl)-1,4-dioxaspiro[4.5]decane (7) from (S)-2-(1,4-dioxaspiro[4.5]decan-7-yl)ethan-1-ol (6)

Ph₃P (1400 g) was dissolved in DCM (5500 mL) under a nitrogen atmosphere. The mixture was cooled to 0° C. and Br₂ (300 mL) and pyridine (2.35 L) were added dropwise, respectively. During the addition the temperature was kept below −10° C. Next, the reaction mixture was warmed to ambient temperature and stirred for 30 min. The reaction mixture was cooled to 0° C. and (S)-2-(1,4-dioxaspiro[4.5]decan-7-yl)ethan-1-ol (6, 900 g) dissolved in DCM (3.6 L) was slowly added (the temperature was kept below 4° C.). The reaction mixture was stirred at ambient temperature for 6 h. MeOH (315 mL), to remove traces of bromine, was added and reaction was stirred for 1 h. The reaction mixture was washed once with water (2300 mL), once with saturated aqueous NaHCO₃ (2300 mL) and once with 5% aqueous Na₂S₂O₃ (1100 mL). The combined aqueous phases were washed twice with DCM (2×1350 mL). The combined organic phases were dried over sodium sulfate and solvent was evaporated to obtain crude compound (7). The crude product was mixed with n-hexane (1600 mL) and stirred for 3 h, filtered and evaporated to obtain (R)-7-(2-bromoethyl)-1,4-dioxaspiro[4.5]decane (7, 1100 g, 91% yield from 6) as a yellow oil.

¹H NMR (400 MHz, CDCl₃) δ 3.92 (m, 4H), 3.40 (t, J=7.0 Hz, 2H), 1.79 (m, 3H), 1.71 (m, 4H), 1.51 (m, 1H), 1.41 (m, 1H), 1.17 (m, 1H), 0.87 (m, 1H) ppm

¹³C NMR (100 MHz, CDCl₃) δ 108.9, 64.3, 64.2, 41.0, 39.7, 34.8, 34.2, 31.2, 31.0, 22.9 ppm

### Example 2

### Preparation of (R)-3-(2-bromoethyl)cyclohexan-1-one (8) from (R)-7-(2-bromoethyl)-1,4-dioxaspiro[4.5]decane (7)

(R)-7-(2-Bromoethyl)-1,4-dioxaspiro[4.5]decane (7, 1100 g) was dissolved in acetonitrile (17,600 mL) under nitrogen atmosphere, and 1 M HCl (17600 mL) was added. Reaction mixture was stirred at ambient temperature overnight. Then acetonitrile was evaporated and the water phase was extracted four times with MTBE (4×4000 mL). The combined organic phases were washed once with saturated aqueous NaHCO₃ (2000 mL), and once with brine (2000 mL). Organic phase was dried over sodium sulfate. After solvent evaporation crude (R)-3-(2-bromoethyl)cyclohexan-1-one (8, 760 g, 84% yield from 7) as a light yellow oil was obtained.

¹H NMR (400 MHz, CDCl₃) δ 3.41 (m, 2H), 2.44 (m, 1H), 2.37 (m, 1H), 2.26 (m, 1H), 2.05 (m, 3H), 1.88 (m, 3H), 1.69 (m, 1H), 1.36 (m, 1H) ppm

¹³C NMR (100 MHz, CDCl₃) δ 210.8, 47.2, 41.3, 39.1, 37.2, 30.5, 24.9 ppm

### Example 3

### Preparation of (R)-3-(2-bromoethyl)cyclohexan-1-one (9) from (R)-3-(2-bromoethyl)cyclohexan-1-one (8)

Ph₃P (146 g), B₂(pin)₂ (1130 g), CuI (141 g) and MeOLi (211 g) were suspended in DMF (12400 mL) under a nitrogen atmosphere. Then, the solution of crude (R)-3-(2-bromoethyl)cyclohexan-1-one (8, 760 g) in DMF (2800 mL) was added. The reaction temperature has slowly increased to 48° C. and the mixture was stirred for 3 h. The dark grey solution was filtered through the Celite pad. To the filtrate, MTBE (7600 mL) and 1 M HCl (2000 mL) were added and aqueous layer was extracted four times with MTBE (4×7600 mL). The combined organic phases were washed four times with brine (4×7600 mL) and dried over sodium sulfate. After evaporating the solvents, the crude compound 9 was obtained. After adding n-hexane (15200 mL) to the residue, the precipitate was formed. The mixture was stirred for 30 min. The resulting precipitate was filtered off and the filtrate was concentrated to afford crude compound 9. (R)-3-(2-Bromoethyl)cyclohexan-1-one (9, 500 g, 53% yield from 8, purity 94% by GC-FID) as a light yellow oil was obtained after distillation under reduced pressure (0.02 mmHg) at about 120° C.

¹H NMR (400 MHz, CDCl₃) δ 2.41 (m, 1H), 2.32 (m, 1H) 2.23 (m, 1H), 2.03 (m, 1H), 1.96 (m, 1H), 1.89 (m, 1H), 1.65 (m, 2H), 1.43 (m, 2H), 1.28 (m, 1H), 1.22 (s, 12H), 0.76 (dd, J₁=9.0 Hz, J₂=7.6 Hz, 2H) ppm

¹³C NMR (100 MHz, CDCl₃) δ 212.3, 83.2, 48.1, 41.6, 41.3, 30.9, 30.8, 25.4, 24.9, 8.1 ppm

### Example 4

Preparation of methyl (4R)-2-oxo-4-(2-(4,4,5,5-tetramethyl-1,3,2-dioxaborolan-2-yl)ethyl)cyclohexane-1-carboxylate (10) from (R)-3-(2-(4,4,5,5-tetramethyl-1,3,2-dioxaborolan-2-yl)ethyl)cyclohexan-1-one (9)

The suitable reactor was charged with sodium hydride (190 g, 60% dispersion in mineral oil) under an argon atmosphere. Then, cyclohexane (400 mL) was added and the obtained suspension was stirred for 20 min at ambient temperature. Then, the solvent was removed and new portion of cyclohexane (400 mL) was added. Procedure was repeated once again. After removing the solvent, dimethyl carbonate (400 mL) was added and the resulting mixture was stirred at ambient temperature for at least 20 min. Dimethyl carbonate was removed, and the fresh dimethyl carbonate (3200 mL), DMF (800 mL) and (R)-3-(2-(4,4,5,5-tetramethyl-1,3,2-dioxaborolan-2-yl)ethyl)cyclohexan-1-one (9, 400 g) were added. The reaction mixture was heated to 75° C. during 60 min. The mixture was stirred at 75° C. for 3 h. Then, the reaction mixture was cooled to ambient temperature, and saturated aqueous NH₄Cl (1600 mL) was added slowly during 40 min. Then, water (800 mL) was added and layers were separated. An aqueous layer was then extracted with MTBE (1600 mL). The combined organic layers were then washed with water (4×1200 mL). The organic layers were concentrated and then evaporated with 2,2,2-trifluoroethanol (400 mL) to give the crude (4R)-2-oxo-4-(2-(4,4,5,5-tetramethyl-1,3,2-dioxaborolan-2-yl)ethyl)cyclohexane-1-carboxylate (10, 478 g, 97% yield) as an orange oil.

¹H NMR (400 MHz, CDCl₃) δ 12.09 (s, 1H), 3.74 (s, 3H), 2.38-2.29 (m, 2H), 2.14-2.07 (m, 1H), 2.01-1.88 (m, 1H), 1.82-1.75 (m, 1H), 1.60-1.51 (m, 1H), 1.45-1.38 (m, 2H), 1.24 (s, 12H), 1.19-1.07 (m, 1H), 0.83-0.73 (m, 2H) ppm

¹³C NMR (100 MHz, CDCl₃) δ 173.0, 171.9, 97.4, 83.0, 51.3, 35.4, 35.2, 29.9, 28.1, 24.8, 22.0 ppm

FT-IR (ATR): 2978, 2926, 2859, 1746, 1715, 1657, 1618, 1443, 1369, 1321, 1273, 1215, 1144, 1051, 968, 843 cm⁻¹

### Example 5

Preparation of methyl (1R,2R,4R)-2-acetamido-2-(tert-butylcarbamoyl)-4-(2-(4,4,5,5-tetramethyl-1,3,2-dioxaborolan-2-yl)ethyl)cyclohexane-1-carboxylate (11) from methyl (4R)-2-oxo-4-(2-(4,4,5,5-tetramethyl-1,3,2-dioxaborolan-2-yl)ethyl)cyclohexane-1-carboxylate (10)

The suitable reactor was charged with the crude methyl (4R)-2-oxo-4-(2-(4,4,5,5-tetramethyl-1,3,2-dioxaborolan-2-yl)ethyl)cyclohexane-1-carboxylate (10, 478 g) in CF₃CH₂OH (3770 mL) under an argon atmosphere. Then, ammonium acetate (368 g) was added at ambient temperature. The obtained mixture was then stirred at ambient temperature for at least 30 min, until all of NH₄OAc was dissolved. Then, the reaction mixture was cooled to 0° C. and t-BuNC (216 ml) was added in one portion. The mixture was stirred at 0° C. for 48 h. Then, the reaction mixture was concentrated to ˜⅕ of initial volume in vacuo and n-hexane (1 L) was added. The obtained mixture was stirred for 10 min and concentrated in vacuo. The obtained mixture was loaded onto silica gel column (800 g in n-hexane) for filtration. The compound 11 was eluted with DCM/MTBE 1:1 (vol./vol., 10 L), the fractions containing product were combined and evaporated to give the crude product (˜700 g).

The crude product 11 was dissolved in i-Pr₂O (1200 mL) under an argon atmosphere. The resulting mixture (as a clear orange solution) was heated to 50° C. and stirred for 60 min. After this time the mixture was slowly cooled to 0° C. and stirred at this temperature overnight. Then, the suspension was cooled to −20° C. and stirred for 3 h. The suspension was then filtrated and the obtained residue was washed with cold i-Pr₂O (400 mL). (1R,2R,4R)-2-acetamido-2-(tert-butylcarbamoyl)-4-(2-(4,4,5,5-tetramethyl-1,3,2-dioxaborolan-2-yl)ethyl)cyclohexane-1-carboxylate (11, 231 g, 32% yield from 9, 97% purity by HPLC) was obtained as white crystals.

¹H NMR (400 MHz, CDCl₃) δ 8.19 (s, 1H), 7.63 (s, 1H), 3.73 (s, 3H), 3.29 (d, J=13.2 Hz, 1H), 2.25 (dd, J₁=12.4 Hz, J₂=4.4 Hz, 1H), 2.14-2.06 (m, 1H), 2.03-1.92 (m, 1H), 1.98 (s, 3H), 1.79 (br d, J=13.0 Hz, 2H), 1.38-1.30 (m, 2H), 1.28 (s, 9H), 1.22 (s, 12H), 1.17-1.06 (m, 2H), 0.91-0.68 (m, 3H) ppm

¹³C NMR (100 MHz, CDCl₃) δ 176.2, 171.2, 170.3, 83.1, 65.8, 52.4, 51.0, 48.5, 40.9, 35.7, 31.2, 30.8, 28.8, 25.3, 25.0, 25.0, 29.4, 8.4 ppm

FT-IR (ATR): 3354, 3304, 3223, 3055, 2959, 2928, 2870, 1711, 1682, 1659, 1568, 1547, 1481, 1449, 1412, 1377, 1362, 1317, 1283, 1217, 1175, 1146, 1119, 1103, 1078, 1028, 1009, 966, 949, 924, 893, 870, 843, 822, 712 cm⁻¹

### Example 6

Preparation of (1R,2S,5R)-1-acetamido-N-(tert-butyl)-2-[(dimethylamino)methyl]-5-(2-(4,4,5,5-tetramethyl-1,3,2-dioxaborolan-2-yl)ethyl)cyclohexane-1-carboxamide (5) from methyl (1R,2R,4R)-2-acetamido-2-(tert-butylcarbamoyl)-4-(2-(4,4,5,5-tetramethyl-1,3,2-dioxaborolan-2-yl)ethyl)cyclohexane-1-carboxylate (11)—Batch Synthesis

Methyl (1R,2R,4R)-2-acetamido-2-(tert-butylcarbamoyl)-4-(2-(4,4,5,5-tetramethyl-1,3,2-dioxaborolan-2-yl)ethyl)cyclohexane-1-carboxylate (11, 15 g) was dissolved in DCM (150 mL) and cooled to −75° C., under an argon atmosphere. Then, 1 M solution of DIBAL-H in DCM (106 mL) was added slowly during 30 min. The mixture was then transferred to another flask containing the cold mixture (−75° C.) of AcOH (9.9 mL), THF (10 mL) and DCM (50 mL). After 3 min of stirring 2 M solution of dimethylamine in THF (44.2 mL) was added during 5 min. The mixture was warmed up to −20° C. and NaBH(OAc)₃ (21.55 g) was added to the reaction mixture in one portion. The mixture was warmed up to ambient temperature and stirred for 2 h. The reaction was quenched with saturated aqueous Na₂CO₃ (200 mL) and stirred for 30 min at ambient temperature. Then, H₂O (150 mL) was added for extraction and the layers were separated. The aqueous phase was extracted twice with DCM (2×100 mL). The combined organic phases were dried over anhydrous Na₂SO₄, filtrated and evaporated to dryness to give the crude (1R,2S,5R)-1-acetamido-N-(tert-butyl)-2-[(dimethylamino)methyl]-5-(2-(4,4,5,5-tetramethyl-1,3,2-dioxaborolan-2-yl)ethyl)cyclohexane-1-carboxamide (5, 11.9 g, 79% yield, 64% purity by HPLC) as a colorless oil.

¹H NMR (400 MHz, CDCl₃) δ 10.21 (s, 1H), 8.26 (s, 1H), 3.40 (dd, J₁=13.4 Hz, J₂=10.4 Hz, 1H), 3.18 (dt, J₁=13.1 Hz, J₂=2.5 Hz, 1H), 2.23 (s, 6H), 2.07 (dd, J₁=13.1 Hz, J₂=4.2 Hz, 1H), 1.95 (dd, J₁=13.5 Hz, J₂=1.6 Hz, 1H), 1.88 (s, 3H), 1.76 (br d, J=12.0 Hz, 1H), 1.53-1.44 (m, 2H), 1.34-1.31 (m, 2H), 1.30 (s, 9H), 1.22 (s, 12H), 0.99-0.88 (m, 2H), 0.84-0.66 (m, 3H) ppm

¹³C NMR (100 MHz, CDCl₃) δ 172.8, 170.8, 82.9, 65.4, 63.6, 50.4, 45.3, 43.3, 42.2, 35.1, 32.5, 30.8, 28.7, 28.1, 24.8, 24.8, 24.5, 8.3 ppm

### Example 7

Preparation of (1R,2S,5R)-1-acetamido-N-(tert-butyl)-2-[(dimethylamino)methyl]-5-(2-(4,4,5,5-tetramethyl-1,3,2-dioxaborolan-2-yl)ethyl)cyclohexane-1-carboxamide (5) from methyl (1R,2R,4R)-2-acetamido-2-(tert-butylcarbamoyl)-4-(2-(4,4,5,5-tetramethyl-1,3,2-dioxaborolan-2-yl)ethyl)cyclohexane-1-carboxylate (11)—In-Flow Synthesis

The suitable reactor was charged with sodium triacethoxyborohydride (NaBH(OAc)₃, 108 g) and DCM (600 mL) and cooled to −76° C. To this suspension, a stream of the combined reagents was fed for 147 min (11/DCM stream: 6.79 mL/min, Me₂NH/AcOH/THF stream: 3.86 mL/min, DIBAL-H/DCM stream: 4.81 mL/min, pipe reactor, cooling bath temperature: 0° C.). During the addition the temperature inside the batch reactor rose to −62° C. After the addition the cooling bath was removed and the obtained mixture was stirred for 2 h at ambient temperature.

The reaction was quenched with 20% aqueous Na₂CO₃ (1000 mL) during 0.5 h and the resulting mixture was stirred for 0.5 h. The obtained suspension was then filtered through the Celite (only the organic layer was filtered). To recover the product from an aqueous layer, the aqueous phase was extracted with DCM (500 mL). The combined organic layers were evaporated to give (1R,2S,5R)-1-acetamido-N-(tert-butyl)-2-[(dimethylamino)methyl]-5-(2-(4,4,5,5-tetramethyl-1,3,2-dioxaborolan-2-yl)ethyl)cyclohexane-1-carboxamide (5, 97 g, 98% yield, 62% purity by HPLC) as a colorless oil.

¹H NMR (400 MHz, CDCl₃) δ 10.21 (s, 1H), 8.26 (s, 1H), 3.40 (dd, J₁=13.4 Hz, J₂=10.4 Hz, 1H), 3.18 (dt, J₁=13.1 Hz, J₂=2.5 Hz, 1H), 2.23 (s, 6H), 2.07 (dd, J₁=13.1 Hz, J₂=4.2 Hz, 1H), 1.95 (dd, J₁=13.5 Hz, J₂=1.6 Hz, 1H), 1.88 (s, 3H), 1.76 (br d, J=12.0 Hz, 1H), 1.53-1.44 (m, 2H), 1.34-1.31 (m, 2H), 1.30 (s, 9H), 1.22 (s, 12H), 0.99-0.88 (m, 2H), 0.84-0.66 (m, 3H) ppm

¹³C NMR (100 MHz, CDCl₃) δ 172.8, 170.8, 82.9, 65.4, 63.6, 50.4, 45.3, 43.3, 42.2, 35.1, 32.5, 30.8, 28.7, 28.1, 24.8, 24.8, 24.5, 8.3 ppm

### Example 8

Preparation of (1R,2S,5R)-1-amino-5-[2-(dihydroxyboranyl)ethyl]-2-[(dimethylamino)methyl]cyclohexane-1-carboxylic acid (1) from (1R,2S,5R)-1-acetamido-N-(tert-butyl)-2-[(dimethylamino)methyl]-5-(2-(4,4,5,5-tetramethyl-1,3,2-dioxaborolan-2-yl)ethyl)cyclohexane-1-carboxamide (5)

(1R,2S,5R)-1-Acetamido-N-(tert-butyl)-2-[(dimethylamino)methyl]-5-(2-(4,4,5,5-tetramethyl-1,3,2-dioxaborolan-2-yl)ethyl)cyclohexane-1-carboxamide (5) (97 g) was dissolved in MTBE (1000 mL) and cooled to 5° C. The resulting mixture was extracted with pre-cooled (5° C.) 0.5 M HCl solution (3×150 mL). The combined aqueous fractions were washed with MTBE (330 mL) and an aqueous phase was transferred to the glass-jacketed reactor (2 L), and 12 M HCl (460 mL) was added in one portion. The reaction mixture was stirred at 100° C. for 2.5 h. The resulting solution was concentrated in vacuo to dryness to give the crude product (63.6 g, 83% yield from 5, 76% purity by UHPLC-FLD) as a brown oil.

The resulting crude hydrochloride of 1 was dissolved in water (250 mL) and the resulting mixture was loaded onto ion-exchange resin column which was prepared from DOWEX® 50WX8 (200-400 mesh, 300 g) and regenerated by 1 M HCl solution and water. The column was eluted with water until pH=7. The column was then eluted with 1 M aqueous NH₃. The fractions containing 1 (based on TLC, 40% MeOH/CHCl₃, KMnO₄ stain) were collected. The solvents were evaporated to give the crude 1 (36.3 g, 60% yield from 5, 82% purity by UHPLC-FLD).

The crude 1 was dissolved in water (100 mL) and stirred at 50° C. for 1 h. Acetone (78.6 g) was slowly added during 0.5 h at 50° C. The resulting mixture was then cooled to 0° C. and agitated for 1 h. The resulting suspension was filtered and the precipitate was washed with pre-cooled (3° C.) acetone (186 mL). After drying the white solid was obtained (26.1 g, 43% yield from 5, 98.9% purity by UHPLC-FLD).

### Example 9

### Recrystallization of (1R,2S,5R)-1-amino-5-[2-(dihydroxyboranyl)ethyl]-2-[(dimethylamino)methyl]cyclohexane-1-carboxylic acid (1)

A suitable reactor was charged with the crude 1 (26.1 g), water (78 mL) and methanol (351 g). The mixture was heated to 65° C. and agitated for 0.5 h. The mixture was concentrated at 85° C. to ½ of initial volume. Acetone (103 g) was then added at 60° C. The mixture was concentrated at 80° C. to ½ of initial volume. Then, acetone (103 g) was added at 50° C. over 10 min. The mixture was concentrated at 80° C. to ½ of initial volume. Then, acetone (205 g) was added at 50° C. during 30 min. The mixture was concentrated at 80° C. to 9/10 of initial volume. Then, acetone (144 g) was added at 50° C. during 30 min. The mixture was then cooled to 0° C. and agitated for 1 h at 0° C. The resulting suspension was filtered, washed with pre-cooled (3° C.) acetone (103 g) and dried to give 1 as a white crystalline material (23.5 g, 39% yield from 5, 99.75% purity by UHPLC-FLD).

Optical rotation: [α]D²⁰=+33.7° (c 1.0, H₂O)

¹H NMR (400 MHz, D₂O) δ 2.92 (dd, J₂=13.1 Hz, J₂=9.5 Hz, 1H), 2.67 (dd, J₁=13.2 Hz, J₂=4.8 Hz, 1H), 2.51 (s, 6H), 2.08-2.03 (m, 1H), 1.85-1.77 (m, 2H), 1.70-1.54 (m, 3H), 1.31-1.25 (m, 2H), 1.07 (t, J=12.6 Hz, 1H), 0.93 (dq, J₁=13.0 Hz, J₂=4.5 Hz, 1H), 0.75 (˜t, J=8.2 Hz, 2H) ppm

¹³C NMR (100 MHz, D₂O) δ 177.6, 63.3, 62.3, 43.7, 43.6, 39.3, 35.1, 30.8, 30.6, 26.4, 11.4 ppm

FT-IR (ATR): 3134, 2992, 2918, 2859, 2826, 2789, 2764, 2727, 1584, 1522, 1458, 1404, 1369, 1344, 1310, 1263, 1202, 1159, 1105, 1074, 1028, 993, 887, 845, 762, 731 cm⁻¹

What is claimed is:

1. A process for the preparation of methyl (1R,2R,4R)-2-acetamido-2-(tert-butyl-carbamoyl)-4-(2-(4,4,5,5-tetramethyl-1,3,2-dioxaborolan-2-yl)ethyl)cyclohexane-1-carboxylate of formula 11 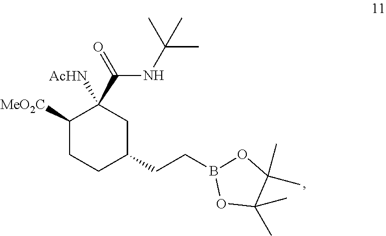 [SMILES: COC(=O)[C@@H]1CC[C@@H](CCB2OC(C)(C)C(C)(C)O2)C[C@]1(NC(C)=O)C(=O)NC(C)(C)C]
the process comprising:
a) reacting (S)-2-(1,4-dioxaspiro[4.5]decan-7-yl)ethan-1-ol of formula 6 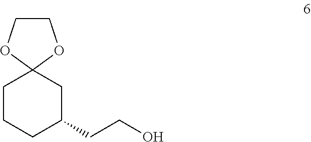 [SMILES: OCC[C@@H]1CCCC2(C1)OCCO2]
with bromine and triphenylphosphine in the presence of an organic base in a reaction solvent at room temperature, followed by quenching the reaction mixture and washing with water, saturated aqueous NaHCO₃, and aqueous Na₂S₂O₃, followed by precipitating triphenylphosphine using a non-polar solvent, drying, and evaporating solvents to provide crude (R)-7-(2-bromoethyl)-1,4-dioxaspiro[4.5]decane of formula 7 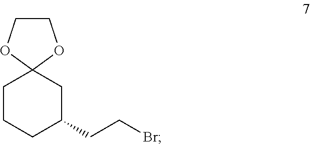 [SMILES: BrCC[C@@H]1CCCC2(C1)OCCO2]
b) reacting the compound of formula 7 dissolved in a reaction solvent with aqueous HCl at ambient temperature, followed by removing the reaction solvent in vacuo, extracting the aqueous phase with an organic solvent, washing the organic phase with saturated aqueous NaHCO₃, and brine, and evaporating solvents to provide crude (R)-3-(2-bromoethyl)cyclohexan-1-one of formula 8 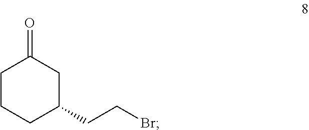
c) reacting the compound of formula 8 dissolved in the first solvent with a mixture of triphenylphosphine, B₂(pin)₂, CuI, and MeOLi suspended in the second solvent, at elevated temperature, followed by filtering, adding the third solvent and aqueous HCl, followed by phase separation, extracting the aqueous phase with the fourth solvent, washing the extract with brine, evaporating solvents, precipitating triphenylphosphine using a non-polar solvent, drying, evaporating volatiles, and distillation under reduced pressure, to provide (R)-3-(2-(4,4,5,5-tetramethyl-1,3,2-dioxaborolan-2-yl)ethyl)-cyclohexan-1-one of formula 9 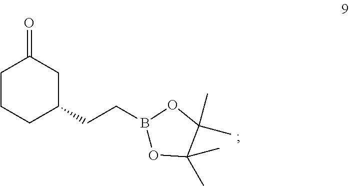 [SMILES: CC1(C)OB(CC[C@@H]2CCCC(=O)C2)OC1(C)C]
d) reacting the compound of formula 9 with dimethyl carbonate, in the presence of a strong base in a solvent at elevated temperature, followed by quenching the reaction with saturated aqueous NH₄Cl, and extraction with a solvent, to provide methyl (4R)-2-oxo-4-(2-(4,4,5,5-tetramethyl-1,3,2-dioxaborolan-2-yl)ethyl)cyclohexane-1-carboxylate of formula 10 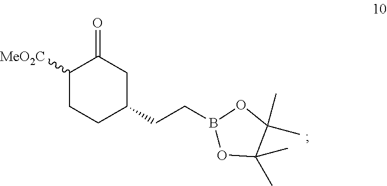 [SMILES: COC(=O)C1CC[C@@H](CCB2OC(C)(C)C(C)(C)O2)CC1=O]
e) dissolving the compound of formula 10 in a solvent, adding ammonium acetate, cooling the solution, and treating with tert-butyl isocyanide to afford the crude compound of formula 11;
f) purifying the crude compound of formula 11 by crystallization from an ether solvent.

2. The process of claim 1, wherein in the step (a) the organic base is a nitrogen heterocycle, preferably pyridine.

3. The process of claim 1, wherein in the step (a) the reaction solvent is DCM.

4. The process of claim 1, wherein in the step (a) the quenching reagent is an alcohol, preferably methanol.

5. The process of claim 1, wherein in the step (a) the non-polar solvent is an alkane, preferably n-hexane.

6. The process of claim 1, wherein in the step (b) the reaction solvent is acetonitrile.

7. The process of claim 1, wherein in the step (b) the organic solvent is an ether, preferably MTBE.

8. The process of claim 1, wherein in the step (c) the first solvent and the second solvent is DMF.

9. The process of claim 1, wherein in the step (c) the elevated temperature is 40° C.

10. The process of claim 1, wherein in the step (c) the third solvent and the fourth solvent is an ether, preferably MTBE.

11. The process of claim 1, wherein in the step (c) the non-polar solvent is an alkane, preferably n-hexane.

12. The process of claim 1, wherein in the step (d) the strong base is sodium hydride.

13. The process of claim 1, wherein in the step (d) the reaction solvent is DMF.

14. The process of claim 1, wherein in the step (d) the reaction temperature does not exceed 75° C.

15. The process of claim 1, wherein in the step (d) the extraction solvent is MTBE.

16. The process of claim 1, wherein in the step (e) the reaction solvent is 2,2,2-trifluoroethanol.

17. The process of claim 1, wherein in the step (e) the reaction temperature is 0° C.

18. The process of claim 1, wherein in the step (f) the ether solvent is i-Pr₂O.

19. A process for the preparation of the solid crystalline (1R,2S,5R)-1-amino-5-[2-(dihydroxyboranyl)ethyl]-2-[(dimethylamino)methyl]cyclohexane-1-carboxylic acid of formula 1 in anhydrous form A 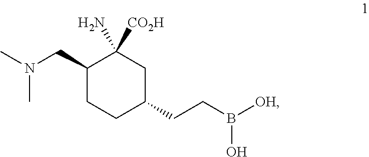 [SMILES: CN(C)C[C@@H]1CC[C@@H](CCB(O)O)C[C@]1(N)C(=O)O]
the process comprising:
a) reacting (S)-2-(1,4-dioxaspiro[4.5]decan-7-yl)ethan-1-ol of formula 6 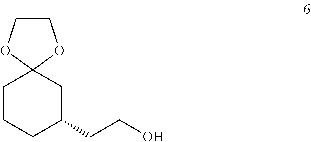 [SMILES: OCC[C@@H]1CCCC2(C1)OCCO2]
with bromine and triphenylphosphine in the presence of an organic base in a reaction solvent at room temperature, followed by quenching the reaction mixture and washing with water, saturated aqueous NaHCO₃, and aqueous Na₂S₂O₃, followed by precipitating triphenylphosphine using a non-polar solvent, drying, and evaporating solvents to provide crude (R)-7-(2-bromoethyl)-1,4-dioxaspiro[4.5]decane of formula 7 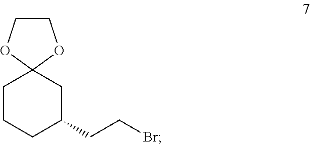 [SMILES: BrCC[C@@H]1CCCC2(C1)OCCO2]
b) reacting the compound of formula 7 dissolved in a reaction solvent with aqueous HCl at ambient temperature, followed by removing the reaction solvent in vacuo, extracting the aqueous phase with an organic solvent, washing the organic phase with saturated aqueous NaHCO₃, and brine, and evaporating solvents to provide crude (R)-3-(2-bromoethyl)cyclohexan-1-one of formula 8 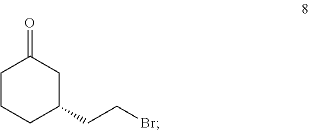
c) reacting the compound of formula 8 dissolved in the first solvent with a mixture of triphenylphosphine, B₂(pin)₂, CuI, and MeOLi suspended in the second solvent, at elevated temperature, followed by filtering, adding the third solvent and aqueous HCl, followed by phase separation, extracting the aqueous phase with the fourth solvent, washing the extract with brine, evaporating solvents, precipitating triphenylphosphine using a non-polar solvent, drying, evaporating volatiles, and distillation under reduced pressure, to provide (R)-3-(2-(4,4,5,5-tetramethyl-1,3,2-dioxaborolan-2-yl)ethyl)-cyclohexan-1-one of formula 9 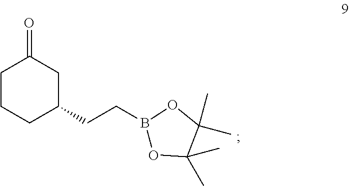 [SMILES: CC1(C)OB(CC[C@@H]2CCCC(=O)C2)OC1(C)C]
d) reacting the compound of formula 9 with dimethyl carbonate, in the presence of a strong base in a solvent at elevated temperature, followed by quenching the reaction with saturated aqueous NH₄Cl, and extraction with a solvent, to provide methyl (4R)-2-oxo-4-(2-(4,4,5,5-tetramethyl-1,3,2-dioxaborolan-2-yl)ethyl)cyclohexane-1-carboxylate of formula 10 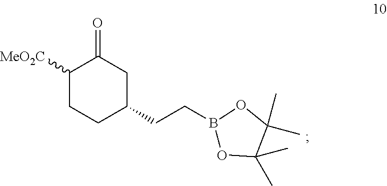 [SMILES: COC(=O)C1CC[C@@H](CCB2OC(C)(C)C(C)(C)O2)CC1=O]
e) dissolving the compound of formula 10 in a solvent, adding ammonium acetate, cooling the solution, and treating with tert-butyl isocyanide to afford the crude compound of formula 11; 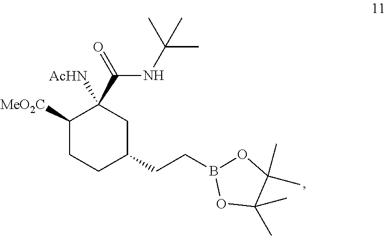 [SMILES: COC(=O)[C@@H]1CC[C@@H](CCB2OC(C)(C)C(C)(C)O2)C[C@]1(NC(C)=O)C(=O)NC(C)(C)C]
f) purifying the crude compound of formula 11 by crystallization from an ether solvent;
g) reacting the compound of formula 11 with DIBAL-H in DCM at a reduced temperature, and quenching the reaction with an organic acid;
h) treating the reaction mixture with dimethylamine at a reduced temperature;
i) treating the reaction mixture with a reducing agent, and quenching the reaction with saturated aqueous sodium carbonate;
j) extracting the reaction mixture with a solvent, to afford crude (1R,2S,5R)-1-acetamido-N-(tert-butyl)-2-[(dimethylamino)methyl]-5-(2-(4,4,5,5-tetramethyl-1,3,2-dioxaborolan-2-yl)ethyl)cyclohexane-1-carboxamide of formula 5 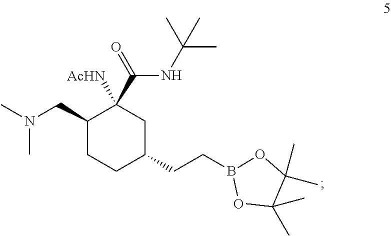
k) dissolving the crude compound of formula 5 in an ether solvent, and extracting the mixture into aqueous HCl;
l) refluxing the extract in aqueous 6 M HCl, cooling down, and evaporating in vacuo;
m) purifying the residue on an ion-exchange resin column, eluting with aqueous ammonia, and evaporating in vacuo, to yield the crude product of formula 1;
n) crystallizing the crude product of formula 1 from water/acetone system;
o) recrystallizing the partially purified product of formula 1 from (water+methanol)/acetone system; and
p) drying the crystals to yield the solid crystalline compound of formula 1 in anhydrous form A.

20. The process of claim 19, wherein in the step (a) the organic base is a nitrogen heterocycle, preferably pyridine.

21. The process of claim 19, wherein in the step (a) the reaction solvent is DCM.

22. The process of claim 19, wherein in the step (a) the quenching reagent is an alcohol, preferably methanol.

23. The process of claim 19, wherein in the step (a) the non-polar solvent is an alkane, preferably n-hexane.

24. The process of claim 19, wherein in the step (b) the reaction solvent is acetonitrile.

25. The process of claim 19, wherein in the step (b) the organic solvent is an ether, preferably MTBE.

26. The process of claim 19, wherein in the step (b) the mild base is NaHCO₃.

27. The process of claim 19, wherein in the step (c) the first solvent and the second solvent is DMF.

28. The process of claim 19, wherein in the step (c) the elevated temperature is 40° C.

29. The process of claim 19, wherein in the step (c) the third solvent and the fourth solvent is an ether, preferably MTBE.

30. The process of claim 19, wherein in the step (c) the non-polar solvent is an alkane, preferably n-hexane.

31. The process of claim 19, wherein in the step (d) the strong base is sodium hydride.

32. The process of claim 19, wherein in the step (d) the reaction solvent is DMF.

33. The process of claim 19, wherein in the step (d) the reaction temperature does not exceed 75° C.

34. The process of claim 19, wherein in the step (d) the extraction solvent is MTBE.

35. The process of claim 19, wherein in the step (e) the reaction solvent is 2,2,2-trifluoroethanol.

36. The process of claim 19, wherein in the step (e) the reaction temperature is 0° C.

37. The process of claim 19, wherein in the step (f) the ether solvent is i-Pr₂O.

38. The process of claim 19, wherein in the step (g) the reaction temperature is −75° C.

39. The process of claim 19, wherein in the step (g) the organic acid is AcOH.

40. The process of claim 19, wherein in the step (h) the reaction temperature is −75° C.

41. The process of claim 19, wherein in the step (i) the reducing agent is sodium triacetoxyborohydride.

42. The process of claim 19, wherein in the step (j) the extracting solvent is DCM.

43. The process of claim 19, wherein in the step (k) the ether solvent is MTBE.

44. The process of claim 19, wherein in the step (m) the ion-exchange resin is DOWEX® 50WX8 regenerated with 1 M HCl.

45. The process of claim 19, wherein in the step (m) the aqueous ammonia is 1 M in NH₃.

46. The process of claim 19, wherein the sequence of steps (g), (h), (i) is replaced by the sequence comprising: dissolving sodium triacetoxyborohydride in DCM and cooling to −75° C.; adding under flow conditions a stream of combined compound 11, dimethylamine in THF, acetic acid, and DIBAL-H in DCM, cooled to 0° C.; and warming up the reaction mixture to the ambient temperature.

47. Solid crystalline (1R,2S,5R)-1-amino-5-[2-(dihydroxyboranyl)ethyl]-2-[(dimethylamino)methyl]cyclohexane-1-carboxylic acid of formula 1 in anhydrous form A, that is characterized by at least one of the following:
(i) XRPD peaks at 7.39, 8.55, 9.49, 12.83, 14.71, 16.86, 17.37, 17.88, 19.11, 19.88, 20.55, 21.19, 22.18, 22.87, 23.66, 24.52, 25.73, 26.09, 26.94, 28.19, 28.61, 28.88, 29.69, 30.28, 31.45, 32.02, 33.06, 33.36, 34.05, 34.52, 35.15, 36.20, 37.52, 38.95, 40.54, 41.76;
(ii) IR bands at 3134, 2992, 2918, 2859, 2826, 2789, 2764, 2727, 1584, 1522, 1458, 1404, 1369, 1344, 1310, 1263, 1202, 1159, 1105, 1074, 1028, 993, 887, 845, 762, 731 cm⁻¹;
(iii) DSC trace with two broad endothermic events: onset at 141.9° C. and peak at 173.7° C.; onset at 237.4° C. and peak at 254.1° C.
